(19) United States
(12) Patent Application Publication
(10) Pub. No.: US 2012/0301879 A1
(43) Pub. Date: Nov. 29, 2012

(54) NOVEL USE OF CA-125

(75) Inventors: YOUNG TAE KIM, Seoul (KR); SUN HAENG KIM, Seoul (KR); KI HOON AHN, Seoul (KR)

(73) Assignee: KOREA UNIVERSITY RESEARCH AND BUSINESS FOUNDATION, Seoul (KR)

(21) Appl. No.: 13/481,511

(22) Filed: May 25, 2012

Related U.S. Application Data
(60) Provisional application No. 61/489,962, filed on May 25, 2011.

(51) Int. Cl. G01N 33/566; C12Q 1/68; C07K 16/28; C07H 21/04

(52) U.S. Cl. 435/6.11; 536/24.31; 435/7.21; 436/501; 435/7.92; 530/389.8

(57) ABSTRACT

Provided is novel use of CA-125. As it is discovered that CA-125 level are positively associated with bone mineral density, thereby CA-125 can be used in biomarker to diagnose osteoporosis or osteopenia, or extent of growth plate development.

### CROSS-REFERENCE TO RELATED APPLICATION

This application claims the priority of U.S. Provisional Appl. Ser. No. 61/489,962, filed May 25, 2011, the entire disclosure of which is incorporated herein by reference.

### BACKGROUND

1. Field of the Invention

The present invention relates to novel use of CA-125 and, more specifically, to diagnose osteoporosis or osteopenia, or extent of growth plate development by using CA-125 level as a biomarker.

2. Discussion of Related Art

Osteoporosis is an imbalance in the remodeling process in which bone resorption exceeds bone formation. A growing understanding of this process has shown that factors involved in inflammation are linked with those factors critical for bone physiology and remodeling.

By measuring bone mineral density (BMD) using dual-energy x-ray absorptiometry (DEXA) or monitoring bone markers such as PYD, NTX, CTX, osteocalcin, PIINP, PIICP, HELIX-II, epitope 846, COMP, pentosidine, MMPs, TIMPs, Glc-Ga1-PYD, YKL-40, hyaluronic acid, etc. which is a factor related to collagen and non-collagen degradation in bone and cartilage, has been widely used in diagnosing or monitoring antiresorptive therapy in clinical trials. However, this measurement does not capture all risk factors for fracture. Bone fragility also depends on the morphology, the architecture, and the remodeling of bone, as well as on the quality (properties) of the bone matrix that cannot be readily assessed. In addition, the risk of fracture is also influenced by muscle function, the propensity to fall, and the ability to adapt to such falls.

Pro-inflammatory cytokines, which are critical mediators of inflammatory responses, have also been shown to regulate bone metabolism, even in individuals without immunologic diseases. Moreover, the association of bone loss with systemic inflammation is supported because serum high sensitivity C-reactive protein (hsCRP), a sensitive marker of chronic, low-grade systemic inflammation, is a significant predictor of osteoporotic fractures. Tumor-associated antigens (TAAs), such as carcinoembryonic antigen (CEA), carbohydrate antigen (CA) 19-9, and CA-125, may be expressed by inflammatory leukocytes apart from tumor cells, and in the soluble form, TAAs may be readily detected in the sera of patients with various autoimmune inflammatory diseases. Based on the potential association of TAAs with systemic inflammation, we determined whether or not high-normal TAA levels are associated with a lower bone mineral density (BMD) in otherwise healthy pre- and post-menopausal women.

### SUMMARY OF THE INVENTION

The present invention is directed to determine whether or not high-normal tumor-associated antigen (TAA) levels are associated with a lower bone mineral density (BMD) and then to diagnose osteoporosis or osteopenia, or extent of growth plate development.

In one aspect, there is provided a kit for diagnosing osteoporosis or osteopenia, or extent of growth plate development comprising a probe selectively binding to carbohydrate antigen 125 (CA-125).

In another aspect, there is provided a method for diagnosing osteoporosis or osteopenia, or extent of growth plate development in an individual, said method comprising:
contacting a biological sample from said individual with a probe selectively binding to carbohydrate antigen 125 (CA-125) and determining expression of CA-125,
wherein under-expression of the CA-125, relative to a normal control, is indicative of osteoporosis or osteopenia in the individual; or
if the individual is a child and adolescent, increased level of the CA-125 is indicative of increase of the extent of growth plate development.

### BRIEF DESCRIPTION OF THE DRAWINGS

The above and other objects, features and advantages of the present invention will become more apparent to those of ordinary skill in the art by describing in detail exemplary embodiments thereof with reference to the attached drawings, in which:

FIG. 1 shows flow chart of the experiment enrolment: TAA, Tumor-associated antigen; CA-125, carbohydrate antigen 125; CA 19-9, carbohydrate antigen 19-9; CEA, carcinoembryonic antigen; AFP, alpha fetoprotein;

FIG. 2 shows Serum CA-125, CA 19-9, CEA and AFP levels in normal, osteopenia and osteoporosis patients according to menopausal status: (A) whole patients; (B) pre-menopause; (C) post-menopause. The BMD values were categorized into three groups according to T-score (normal, ≧−1.0; osteopenia, −1.0˜2.5; and osteoporosis, ≦−2.5) according to World Health Organization classification. Lower BMD (osteopenia or osteoporosis) category was used because the number of pre-menopausal women with osteoporosis (n=24) was not enough statistically. Analysis of covariance (ANCOVA) was performed to adjust for age, BMI and menopausal status in whole patients; age and BMI in pre-menopausal women; and age, BMI and menopausal duration in post-menopausal women. *P<0.05 was considered statistically significant. The data were presented as the mean (95% confidence interval) and the P values were the results of ANCOVA. CA-125, carbohydrate antigen 125; CA 19-9, carbohydrate antigen 19-9; CEA, carcinoembryonic antigen; AFP, alpha fetoprotein; BMD, bone mineral density; BMI, body mass index;

FIG. 3 shows positive immunostain in chondrocytes of articular cartilage (A); positive immunostain in chondrocytes of growth plate (B); weak or negative immunostain in osteocytes of trabecular bone (C).

### DETAILED DESCRIPTION OF EXEMPLARY EMBODIMENTS

Hereinafter, the present invention will be described with reference to examples and comparative examples in detail. However, the present invention is not limited to these examples.

The present invention relates to a kit for diagnosing osteoporosis or osteopenia, or extent of growth plate development comprising a probe selectively binding to carbohydrate antigen 125 (CA-125).

CA-125 is a high molecular weight, cell surface glycoprotein detected in the serum of a large proportion of patients with ovarian epithelial cancer (OEC). However, while the percentage is high (75-90%) in advanced stages of this disease, it is only elevated in 50% of the patients with Stage 1 disease. Use of CA-125 as a marker for OEC is problematic because the molecule is also expressed in a number of normal and pathological conditions including menstruation, pregnancy, endometriosis, inflammatory diseases and other types of cancer. Improved sensitivity and specificity for OEC has been reported among post menopausal women. See, for example, Bast et al. (1998) Int'l J. Biological Markers 13:170-187; and Moss et al (2005) J. Chn. Pathol. 58:308-312. In addition, with respect to bone metabolism, its function has not been used or found.

The inventors found a significant positive association between CA-125 and bone mineral density (BMD) of a diagnostic reference of osteoporosis, that is, the serum CA-125 shows lower level in the lower BMD group.

In particular embodiments, CA-125 level in group with osteoporosis or osteopenia is significantly lower than in group with normal BMD. The result of dividing a pre-menopause group into a group with normal BMD and a group with lower BMD, CA-125 level is significantly lower in the group with lower BMD than the group with normal BMD. Also, CA-125 shows significantly the lower level in a post-menopause group with osteoporosis or osteopenia. The CA-125 level in post-menopausal group had a significant inverse relationship with the ALP level, one of the bone turnover markers. And a serum high sensitivity C-reactive protein (hsCRP), a sensitive marker of chronic, low-grade systemic inflammation was not associated with BMD in women without inflammatory and immunologic disorders irrespective of menopausal status and there was no relationship between hsCRP and CA-125. Therefore, it is unlikely that the relationship of CA-125 level within the normal range to bone mass is via an inflammatory process. The CA-125 level would have an estrogen-independent relationship with BMD.

CA-125, which is proportional to BMD, is potentially useful auxiliary marker to BMD in the management of bone loss like serum bone formation and resorption markers.

In immunohistochemical staining of CA-125 in the femur of rat, chondrocytes of articular cartilage and growth plate shows a positive staining and osteocytes of trabecular bone also showed weak or negative staining. Therefore, CA-125 of a biomarker of chondrocytes may be used in diagnosing the extent of growth plate development relation to growth of child and adolescent.

Herein, the term “diagnosis” refers to means the process of knowledge gaining by assigning symptoms or phenomena to a disease or injury. In the present case, the presence or absence of particular marker is also used for differential diagnosis. The presence or absence of a marker can be measured by any method known in the prior art. Methods which may be used are exemplified below. “Diagnosing osteoporosis or osteopenia” is intended to include, for example, diagnosing or detecting the presence of osteoporosis or osteopenia, monitoring the progression of the disease, and identifying or detecting cells or samples that are indicative of osteoporosis or osteopenia. Also, “Diagnosing extent of growth plate development” is intended to include, for example, monitoring the increase of the extent of growth plate development composed of chodrocytes relation to growth of child and adolescent.

The term “diagnosis marker” means organic biological molecules including any polypeptide or nucleic acid (e.g. mRNA etc.), lipid, glycolipid, glycoprotein, saccharides(monosaccharide, disaccharide, oligosaccharide, etc.) etc. whose level of expression in a tissue or cell is altered compared to that of a normal or healthy cell or tissue. Diagnosis marker of the invention is selective for osteoporosis or osteopenia. The term “selectively underexpressed in osteoporosis or osteopenia” is intended that the biomarker of interest is underexpressed in osteoporosis or osteopenia but is not underexpressed in conditions that are not considered to be clinical disease. Thus, detection of the biomarker of the invention permits the differentiation of samples indicative of an increased likelihood of having osteoporosis or osteopenia. Diagnosis marker of the invention may be referred to herein interchangeably as “osteoporosis or osteopenia biomarker”. Reference to “diagnosis marker” herein is a term of convenience to refer to the marker described herein and their use, and is not intended to indicate the marker is only used to diagnose osteoporosis or osteopenia. As this disclosure makes clear, the biomarker is useful for, for example, assessing cognitive function, assessing risk of developing osteoporosis or osteopenia, stratifying osteoporosis or osteopenia, etc.

The term “probe” refers to any molecule that is capable of selectively binding to a specifically intended target biomolecule, for example, a nucleotide transcript or a protein encoded by or corresponding to a biomarker. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules. Isolated mRNA can be used in hybridization or amplification assays that include, but are not limited to, Southern or Northern analyses, polymerase chain reaction analyses and probe arrays.

The expression of a biomarker of interest is detected at the nucleic acid level. Nucleic acid-based techniques for assessing expression are well known in the art and include, for example, determining the level of biomarker mRNA in a biological sample. Many expression detection methods use isolated RNA. Any RNA isolation technique that does not select against the isolation of mRNA can be utilized for the purification of RNA from serum samples (see, e.g., Ausubel et al, ed., (1987-1999) Current Protocols in Molecular Biology (John Wiley & Sons, New York). Additionally, large numbers of blood, serum, or tissue samples can readily be processed using techniques well known to those of skill in the art, such as, for example, the single-step RNA isolation process of Chomczynski (1989, U.S. Pat. No.

4,843,155).

One method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding a biomarker of the present invention. Hybridization of an mRNA with the probe indicates that the biomarker in question is being expressed. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the biomarkers of the present invention. An alternative method for determining the level of biomarker mRNA in a sample involves the process of nucleic acid amplification, e.g., by RT-PCR (the experimental embodiment set forth in Mullis, 1987, U.S. Pat. No. 4,683,202), ligase chain reaction (Barany (1991) Proc. Natl. Acad ScL USA 88:189493), self sustained sequence replication (Guatelli et al. (1990) Proc. Natl. Acad. ScL USA 87:1874-1878), transcriptional amplification system (Kwoh et al. (1989) Proc. Natl Acad. ScL USA 86:1173-1177), Q-Beta Replicase (Lizardi et al. (1988) Bio/Technology 6: 1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, biomarker expression is assessed by quantitative fluorogenic RT-PCR (i.e., the TaqMan® System). Such methods typically utilize pairs of oligonucleotide primers that are specific for the biomarker of interest. Methods for designing oligonucleotide primers specific for a known sequence are well known in the art. Biomarker expression levels of RNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of biomarker expression may also comprise using nucleic acid probes in solution.

Biomarker expression of the invention is detected at the protein level using antibodies. The terms “antibody” and “antibodies” broadly encompass naturally occurring forms of antibodies and recombinant antibodies such as single-chain antibodies, chimeric and humanized antibodies and multi-specific antibodies as well as fragments and derivatives of all of the foregoing, which fragments and derivatives have at least an antigenic binding site. Antibody derivatives may comprise a protein or chemical moiety conjugated to the antibody.

“Antibodies” and “immunoglobulins” (Igs) are glycoproteins having the same structural characteristics. While antibodies exhibit binding specificity to an antigen, immunoglobulins include both antibodies and other antibody-like molecules that lack antigen specificity. Polypeptides of the latter kind are, for example, produced at low levels by the lymph system and at increased levels by myelomas.

The term “antibody” is used in the broadest sense and covers fully assembled antibodies, antibody fragments that can bind antigen (e.g., Fab′, F′(ab) 2, Fv, single chain antibodies, diabodies), and recombinant peptides comprising the foregoing. The term “monoclonal antibody” as used herein refers to an antibody obtained from a population of substantially homogeneous antibodies, i.e., the individual antibodies comprising the population are identical except for possible naturally- occurring mutations that may be present in minor amounts.

“Antibody fragments” comprise a portion of an intact antibody, preferably the antigen-binding or variable region of the intact antibody. Examples of antibody fragments include Fab, Fab′, F(ab′)2, and Fv fragments; diabodies; linear antibodies (Zapata et at. (1995) Protein Eng, 8 (10):1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize 35 readily. Pepsin treatment yields an F(ab′)2 fragment that has two antigen-combining sites and is still capable of cross-linking antigen. “Fv” is the minimum antibody fragment that contains a complete antigen recognition and binding site. In a two-chain Fv species, this region consists of a dimer of one heavy- and one light-chain variable domain in tight, non-covalent association. In a single-chain Fv species, one heavy- and one light-chain variable domain can be covalently linked by flexible peptide linker such that the light and heavy chains can associate in a “dimeric” structure analogous to that in a two-chain Fv species. It is in this configuration that the three CDRs of each variable domain interact to define an antigen-binding site on the surface of the V H-V L dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. The Fab fragment also contains the constant domain of the light chain and the first constant domain (C H I) of the heavy chain. Fab fragments differ from Fab′ fragments by the addition of a few residues at the carboxy terminus of the heavy-chain C H I domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Polyclonal antibodies can be prepared by immunizing a suitable subject (e.g., rabbit, goat, mouse, or other mammal) with a biomarker protein immunogen. The antibody titer in the immunized subject can be monitored over time by standard techniques, such as with an enzyme linked immunosorbent assay (ELISA) using immobilized biomarker protein. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. T/US2007/061205 Today 4:72), the EBV-hybridoma technique (Cole et al. (1985) in Monoclonal Antibodies and Cancer Therapy, ed. Reisfeld and Sell (Alan R. Liss, Inc., New York, N.Y.), pp. 77-96) or trioma techniques. The technology for producing hybridomas is well known {see generally Coligan et ah, eds. (1994) Current Protocols in Immunology (John Wiley & Sons, Inc., New York, N.Y.); Galfre et al (1977) Nature 266:550-52; Kenneth (1980) in Monoclonal Antibodies: A New Dimension In

Biological Analyses (Plenum Publishing Corp., NY); and Lerner (1981) Yale J. Biol. Med., 54:387-402).

Alternative to preparing monoclonal antibody-secreting hybridomas, a monoclonal antibody can be identified and isolated by screening a recombinant combinatorial immunoglobulin library (e.g., an antibody phage display library) with a biomarker protein to thereby isolate immunoglobulin library members that bind the biomarker protein. Kits for generating and screening phage display libraries are commercially available (e.g., the Pharmacia Recombinant Phage Antibody System, Catalog No. 27-9400-01 ; and the Stratagene SurβAPθ Phage Display Kit, Catalog No. 240612). Additionally, examples of methods and reagents particularly amenable for use in generating and screening antibody display library can be found in, for example, U.S. Pat. No. 5,223,409; PCT Publication Nos. WO 92/18619; WO 91/17271; WO 92/20791; WO 92/15679; 93/01288; WO 92/01047; 92/09690; and 90/02809; Fuchs et al. (1991) Bio/Technology 9:1370-1372; Hay et al. (1992) Hum. Antibod Hybridomas 3:81-85; Huse et al. (1989) Science 246:1275-1281; Griffiths et al. (1993) EMBO J. 12:725-734.

The kit of the present invention further comprises monoclonal antibodies and variants and fragments thereof that specifically bind to biomarker protein of interest. The monoclonal antibodies may be labeled with a detectable substance as described below to facilitate biomarker protein detection in the sample. Such antibodies find use in practicing the methods of the invention. Monoclonal antibodies having the binding characteristics of the antibodies disclosed herein are also encompassed by the present invention. Compositions further comprise antigen-binding variants and fragments of the monoclonal antibodies, hybridoma cell lines producing these antibodies, and isolated nucleic acid molecules encoding the amino acid sequences of these monoclonal antibodies.

Antibodies having the binding characteristics of a monoclonal antibody of the invention are also provided. “Binding characteristics” or “binding specificity” when used in reference to an antibody means that the antibody recognizes the same or similar antigenic epitope as a comparison antibody. Examples of such antibodies include, for example, an antibody that competes with a monoclonal antibody of the invention in a competitive binding assay. One of skill in the art could determine whether an antibody competitively interferes with another antibody using standard methods.

By “epitope” is intended the part of an antigenic molecule to which an antibody is produced and to which the antibody will bind. Epitopes can comprise linear amino acid residues (i.e., residues within the epitope are arranged sequentially one after another in a linear fashion), nonlinear amino acid residues (referred to herein as “nonlinear epitopes”; these epitopes are not arranged sequentially), or both linear and nonlinear amino acid residues. Typically epitopes are short amino acid sequences, e.g. about five amino acids in length. Systematic techniques for identifying epitopes are known in the art and are described, for example, in U.S. Pat. No. 4,708,871. Briefly, a set of overlapping oligopeptides derived from the antigen may be synthesized and bound to a solid phase array of pins, with a unique oligopeptide on each pin. The array of pins may comprise a 96-well microliter plate, permitting one to assay all 96 oligopeptides simultaneously, e.g., for binding to a biomarker-specific monoclonal antibody. Alternatively, phage display peptide library kits (New England BioLabs) are currently commercially available for epitope mapping. Using these methods, the binding affinity for every possible subset of consecutive amino acids may be determined in order to identify the epitope that a given antibody binds. Epitopes may also be identified by inference when epitope length peptide sequences are used to immunize animals from which antibodies are obtained. Epitopes may also be defined by carbohydrate side chains present as either N-linked or O-linked oligosaccharides present on glycoproteins.

By the kit of the present invention is intended any manufacture (e.g., a package or a container) comprising at least one reagent, e.g., an antibody, a nucleic acid probe, etc. for specifically detecting the expression of a biomarker of the invention. The kit may be promoted, distributed, or sold as a. unit for performing the methods of the present invention. Additionally, the kit may contain a package insert describing the kit and methods for its use.

Methods for detecting biomarker of the invention comprise any methods that determine the quantity of the biomarker either at the nucleic acid or protein level. Such methods are well known in the art and include but are not limited to western blots, northern blots, southern blots, ELISA, immunoprecipitation, immunofluorescence, flow cytometry, immunocytochemistry, multiplex bead-based immunoassays, nucleic acid hybridization techniques, nucleic acid reverse transcription methods, and nucleic acid amplification methods. In particular embodiments, underexpression of a biomarker is detected on a protein level using, for example, antibodies that are directed against specific biomarker protein. These antibodies can be used in various methods such as Western blot, ELISA, multiplex bead-based immunoassay, immunoprecipitation, or immunocytochemistry techniques. The multiplex bead-based assays used to practice the present invention include but are not limited to the Luminex technology described in U.S. Pat. Nos. 6,599,331, 6,592,822, and 6,268,222, all of which are herein incorporated by reference in their entirety. In particular embodiment, the Luminex LabMAP® system is utilized, as described in International Publication No. WO 2005/016126, which is herein incorporated by reference it its entirety.

Detection of antibody binding can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluoresce in isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H. The antibodies used to practice the invention are selected to have high specificity for the biomarker proteins of interest. Methods for making antibodies and for selecting appropriate antibodies are known in the art. See, for example, Celis, ed. (in press) Cell Biology & Laboratory Handbook, 3rd edition (Academic Press, New York), which is herein incorporated in its entirety by reference. In some embodiments, commercial antibodies directed to specific biomarker proteins may be used to practice the invention. The antibodies of the invention may be selected on the basis of desirable staining of cytological, rather than histological, samples. That is, in particular embodiments the antibodies are selected with the end sample type (i.e., serum samples) in mind and for binding specificity.

The present invention relates to a method for diagnosing osteoporosis or osteopenia, or extent of growth plate development in an individual, said method comprising:
contacting a biological sample from said individual with a probe selectively binding to carbohydrate antigen 125 (CA-125) and determining expression of CA-125,
wherein under-expression of the CA-125, relative to a normal control, is indicative of osteoporosis or osteopenia in the individual; or
if the individual is a child and adolescent, increased level of the CA-125 is indicative of increase of the extent of growth plate development.

Said biological sample is intended any sampling of tissues, cells, blood, serum, plasma, sputum, urine, etc. biological sample may be obtained from a patient by a variety of techniques including, for example, by venipuncture, by scraping or swabbing an area, or by using a needle to aspirate bodily fluids or tissues. Methods for collecting various body samples are well known in the art. In particular embodiments, the biological sample comprises blood or serum.

Methods for detecting biomarker of the invention comprise any methods that determine the quantity of the biomarker either at the nucleic acid or protein level. Such methods are well known in the art.

In one embodiment of the invention, microarrays are used to detect biomarker expression. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample.

Techniques for the synthesis of these arrays using mechanical synthesis methods are described in, e.g., U.S. Pat. No. 5,384,261, incorporated herein by reference in its entirety for all purposes. Although a planar array surface is preferred, the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, each of which is hereby incorporated in its entirety for all purposes. Arrays may be packaged in such a manner as to allow for diagnostics or other manipulation of an all-inclusive device. See, for example, U.S. Pat. Nos. 5,856,174 and 5,922,591 herein incorporated by reference.

In one approach, total rnRNA isolated from the sample is converted to labeled cRNA and then hybridized to an oligonucleotide array. Each sample is hybridized to a separate array. Relative transcript levels may be calculated by reference to appropriate controls present on the array and in the sample.

One of skill in the art will further appreciate that any or all steps in the diagnostic method of the invention could be implemented by personnel or, alternatively, performed in an automated fashion. That is, the methods can be performed in an automated, semi-automated, or manual fashion. Furthermore, the methods disclosed herein can also be combined with other methods known or later developed to permit a more accurate diagnosis of patients having an increased likelihood of having osteoporosis or osteopenia.

Detection of the biomarker of the invention permits the differentiation of samples indicative of an increased likelihood of having osteoporosis or osteopenia. Thus, comparison with its expression level in a normal population, the underexpressed biomarker of the present invention either at nucleic acid or protein level may be assessed to osteoporosis or osteopenia.

Also, in immunohistochemical staining of CA-125 in chodrocytes, detection of the biomarker of the invention permits monitoring the extent of growth plate development composed of chodrocytes relation to growth of child and adolescent.

Hereinafter, the present invention will be described in further detail with respect to examples according to the present invention and comparative examples not according to the present invention, but the scope of the present invention is not limited by the following examples.

### EXAMPLE 1

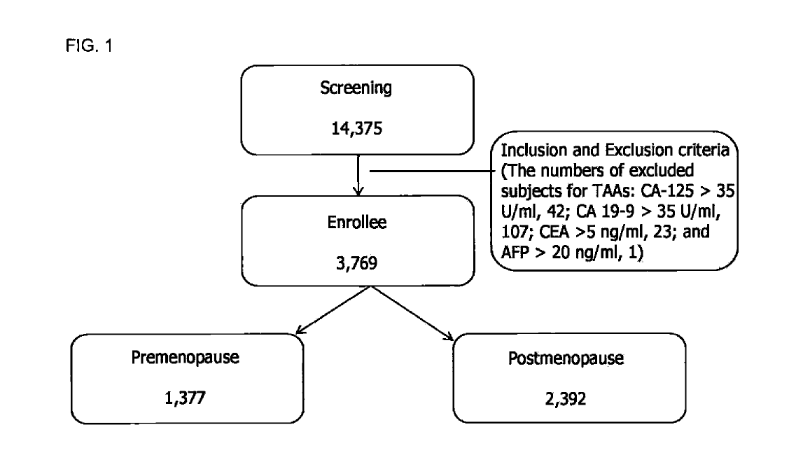

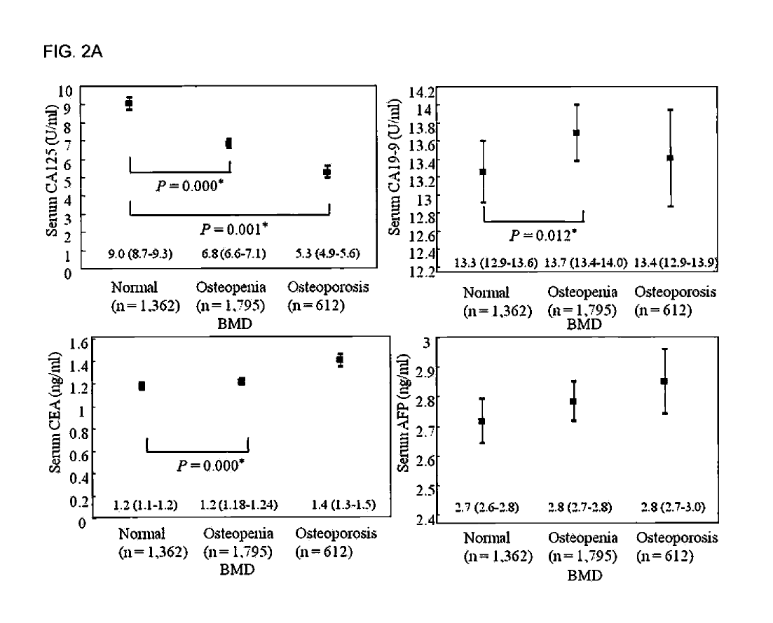

The present experiment was approved by the Institutional Review Board of Korea University Medical Center in Seoul. The study population consisted of 14,375 consecutive Korean women who participated in the health screening program at the Korea University Medical Center between January 2004 and December 2008 (FIG. 1).

Special trainees administered a questionnaire to all subjects to obtain information on their cigarette smoking and alcohol consumption habits, leisure time physical exercise (number of days per week), medication history, history of medical and surgical diseases, and reproductive history. The height (cm) and weight (kg) of each subject were measured in light clothing without shoes and the body mass index (BMI; kg/m²) was calculated. The oral temperature was measured, and a physician examined each patient. A bimanual pelvic examination was performed with a liquid-based cytology test. The following routine laboratory and imaging examinations were obtained: complete blood count; liver, renal, and thyroid function tests; fasting serum concentrations of glucose (mg/dl), insulin (μlU/ml), calcium (mg/dl), phosphorus (mg/dl), total alkaline phosphatase (ALP; U/l), uric acid (mg/dl), rheumatoid factor (RF; IU/ml), hsCRP (mg/l), follicle stimulating hormone (FSH; IU/l), total cholesterol (TC; mg/dl), high-density lipoprotein-cholesterol (HDL-C; mg/dl), low-density lipoprotein-cholesterol (LDL-C; mg/dl), triglycerides (TG; mg/dl), CA-125 (U/ml), CA 19-9 (U/ml), CEA (ng/ml), and AFP (ng/ml); urinalysis; stool occult blood test; simple chest X-ray; gastrofiberoscopy; colonoscopy; abdominal ultrasonography; and mammography.

We excluded women who had the following: a hysterectomy prior to the natural menopause; known thyroid dysfunction, diabetes mellitus, pituitary disease, hypogonadism, chronic liver disease, chronic renal disease, or any diseases which can affect bone metabolism; a history of cancer which can affect TAA levels, as well as uterine myomas, adenomyosis, and endometriosis; use of a bisphosphonate, a selective estrogen receptor modulator, estrogen, calcitonin, thyroid hormone, cortisone, calcium, vitamin D, diuretics, or any drugs which can derange bone metabolism; and a stroke or dementia because of concerns with limited physical activity. Other exclusion criteria were as follows: an oral temperature >38.0° C.; and any abnormal findings on routine laboratory and imaging examinations, including an abnormal leukocyte count (<4.0 or >10.0×10⁹/l), elevated serum aspartate aminotransferase (AST; ≧40 IU/l), alanine aminotransferase (ALT; ≧40 IU/l), or total ALP (>120 U/l) concentrations, abnormal serum calcium (<8.3 or >10.0 mg/dl) or phosphorus (<2.5 or >4.5 mg/dl) concentrations, decreased serum albumin concentration (<3.0 g/dl), elevated serum creatinine (≧1.5 mg/dl) or fasting glucose (≧126 mg/dl) concentrations, abnormal serum free thyroxine (T4; ≦0.8 or ≧1.9 ng/dl) or thyroid stimulating hormone (TSH; ≦50.5 or ≧5.0 mU/l) concentrations, and elevated serum RF (≧20 IU/ml), hsCRP (>10 mg/l), or uric acid (>6 mg/dl) concentrations. Elevated TAA levels (CA-125 >35 U/ml; CA 19-9>35 U/ml; CEA >5 ng/ml; and AFP >20 ng/ml) were excluded to rule out any association with cancer-related bone changes.

A total of 3,769 were enrolled, of whom 1,377 were pre-menopausal and 2,392 were post-menopausal. Post-menopause was defined as amenorrhea for at least 1 year, combined with a serum FSH >30 IU/l. Blood samples were collected between 8:00 and 11:00 AM after at least a 12 h fast from each participant. Serum CA-125, CA 19-9, CEA, and AFP levels were measured by an electrochemiluminescence immunoassay (ECLA) using an E170 (Roche Diagnostics Corporation, Indianapolis, Ind., USA). TSH and T4 levels were also measured using an E170. Serum FSH was measured by an immunoradiometric assay (IRMA) using a Stratec SR 300 (STRATEC Biomedical Systems ACT, Birkenfeld, Germany). Serum calcium, phosphorus, and creatinine levels were measured by colorimetry (Toshiba 200FR; Toshiba, Tokyo, Japan). An E170 was used to measure the serum fasting insulin level. Serum fasting glucose, TC, HDL-C, TG, LDL-C, and ALP were measured by colorimetry (Toshiba 200FR). The intra- and inter-assay coefficients of variance (CVs) of the variables were checked. Dual-energy X-ray absorptiometry (Discovery-W Hologic; Bedford, Mass., USA) was used to measure the BMD of the non-dominant femoral neck and the antero-posterior lumbar spine (L1-L4). The lowest value was used for the analysis and the CV for the BMD was 1.0%. The T-score was calculated. The results of BMD were classified as follows: normal (T-score ≧1.0); osteopenia (−2.5<T-score <−1.0); and osteoporosis (T-score ≦−2.5).

Continuous variables are presented as the means±standard deviation (SD) with a normal distribution. The basic clinical and laboratory characteristics between pre- and post-menopausal women were compared using a Student's t-test. The mean and 95% confidence intervals of serum CA-125, CA 19-9, CEA, and AFP levels were compared by analysis of covariance (ANCOVA) among the different BMD categories. Analysis of variance (ANOVA) was performed to compare the clinical and laboratory data among the quintiles for the significant TAAs based on

ANCOVA. The P for trend was calculated for the linearity among the quintiles. Partial correlation coefficients among serum CA-125, CEA, ALP, and hsCRP levels, and BMD were calculated after the adjustment of age and BMI. All statistical analyses were performed using SAS 9.1.3 (SAS Institute Inc., Cary, N.C., USA) and a P<0.05 was considered statistically significant. A sample size power calculation indicated that 3,769 participants were sufficient to perform the study with a power of 80% and an alpha error of 5%.

### Immunohistochemistry

For indirect immunohistochemistry, the epitopes blocked during fixation and embedding were exposed using the following demasking procedure. After removal of the embedding material, endogenous peroxidase was blocked with H₂O₂, then, semi-thin sections were rinsed in saline and incubated in phosphate buffered citrate solution pH 7.4 (Dako, Glostrup, Denmark) for 20 min in the microwave and rinsed with phosphate buffered saline (PBS). Unspecific binding was blocked with 10% horse serum. Rabbit anti-human CA-125 polyclonal antibody (1:200, Catalog number: 250566, Abbiotec™) as primary antibody was incubated for 8 h at 4° C., afterwards the secondary biotin-conjugated antibody was applied for 30 min. Sections were counterstained with hematoxylin (Merck, Karmstadt, Germany) for 10 second. Between incubation steps saline rinses were performed. The sections were mounted in 40% glycerol gelatin (Merck, Darmstadt, Germay) in PBS.

The demographic and biomedical characteristics of the study subjects are shown in Table 1. The mean duration of menopause in post-menopausal women was 10.5 years.

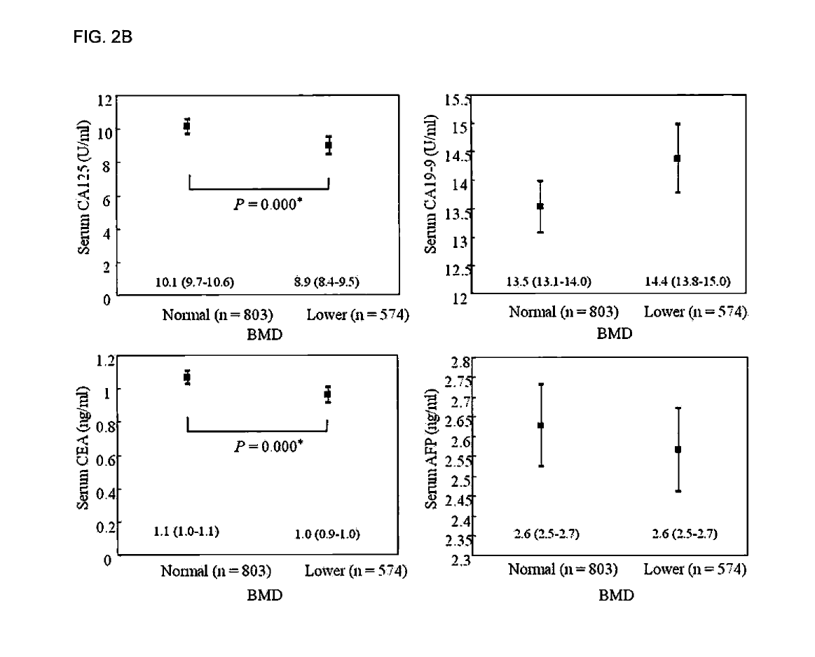

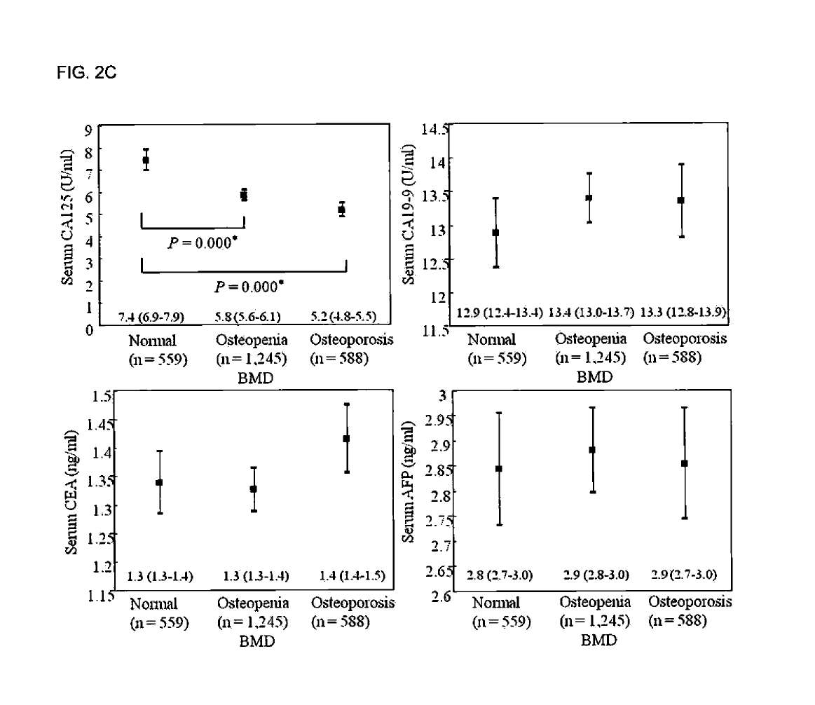

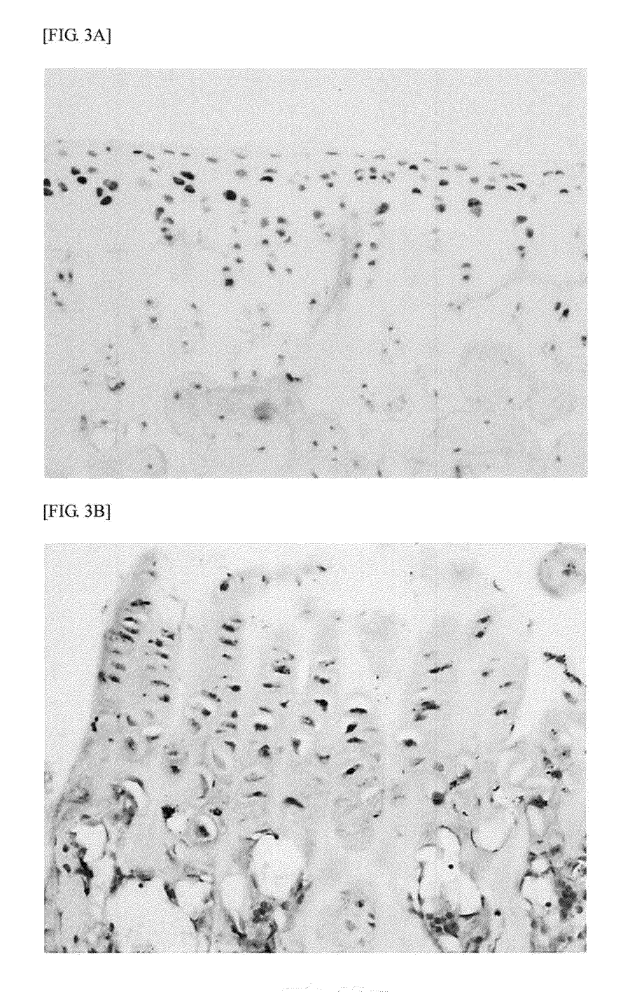

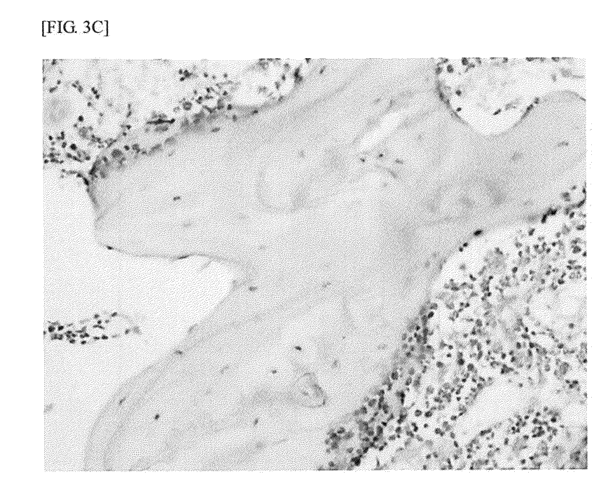

In an analysis of the entire cohort of patients after adjustment for age, BMI, and menopausal status, the CA-125 levels in patients with osteopenia (P<0.001) and osteoporosis (P=0.001) were lower than in patients with a normal BMD (FIG. 2A). The CA 19-9 (P=0.012) and CEA levels (P<0.001) in patients with osteopenia were higher than in patients with a normal BMD. The APP levels were not significantly different among the three BMD groups. The study subjects were divided into pre- and post-menopause groups according to menopausal status to adjust the confounding effect of menopause on the levels of TAAs. The number of osteoporosis patients was only 24 in the pre-menopause group, so the osteoporosis group was combined with the osteopenia group into a lower BMD group. In pre-menopausal women, only the CA-125 (P<0.001) and CEA levels (P<0.001) were lower in the lower BMD group than the normal BMD group after adjusting for age and BMI (FIG. 2B). In post-menopausal women, after adjustment for age, BMI, and duration of menopause, only the CA-125 level was significantly different among patients with a normal BMD, osteopenia, and osteoporosis (P<0.001; FIG. 2C).

CA-125 and CEA were divided into quintiles (Q1, lowest; Q5, highest), and ANOVA with linearity of variables according to the quintiles was performed (Table 2). In pre-menopausal women, serum calcium and phosphorus levels and BMD T-scores were significantly different according to CA-125 quintiles (Table 2A). On analysis of the CA-125 quintiles in post-menopausal women, age, serum ALP, calcium, and phosphorus levels and BMD T-scores had significant differences. On analysis of CEA in pre-menopausal women, age, serum calcium levels, and BMD T-scores were different according to the quintiles (Table 2B).

Based on partial correlation analysis (Table 3), the CA-125 level had a significant correlation with BMD (r=0.102; P<0.001) in the pre-menopause. The CEA level also had a significant correlation with BMD (r=0.134; P<0.001). The CA-125 level was positively correlated with the hsCRP level (r=0.052; P=0.01) and BMD (r=0.104; P<0.001), and negatively correlated with the ALP level (r=−0.298; P<0.001) in the post-menopause.

As a result of sensitivity, specificity, PPV, NPV, and ROC analysis (Table 4), a CA-125 1 9.6 U/ml (P=0.001) and a CEA <0.8 ng/ml (P=0.009) in the pre-menopause, and a CA-125 <8.9 U/ml (P=0.000) in the post-menopause, were significant in discriminating osteopenia and osteoporosis from a normal BMD. The sensitivity and PPV of a CA-125 <8.9 U/ml for the diagnosis of osteopenia and osteoporosis in the post-menopause were 83.3% and 79.4%, respectively.

In immunohistochemical staining of CA-125 in the femur of rat, chondrocytes of articular cartilage and growth plate showed a positive staining and osteocytes of trabecular bone also showed weak or negative staining.

TABLE 1
Basic characteristic of study subjects

| | Pre-menopause | Post-menopause | |
|---|---|---|---|
| | (n = 1377) | (n = 2392) | |
| Variables | Mean ± SD | Mean ± SD | P |
| Age (years) | 43.1 ± 5.8 | 60.5 ± 6.7 | <0.001 |
| Height (cm) | 158.4 ± 5.3 | 155.2 ± 5.3 | <0.001 |
| Weight (kg) | 55.5 ± 7.3 | 57.8 ± 7.3 | <0.001 |
| BMI (kg/m²) | 22.1 ± 2.9 | 24.0 ± 2.8 | <0.001 |
| Physical exercise | 2.0 ± 1.9 | 1.8 ± 1.7 | 0.008 |
| (number of days/week) | | | |
| Current or past smoker | 29 (2.1) | 36 (1.5) | 0.003 |
| (n (%)) | | | |
| Habitual drinker (n (%)) | 264 (19.2) | 361 (15.1) | 0.005 |
| Serum ALP (U/L) | 45.7 ± 11.6 | 61.4 ± 16.9 | <0.001 |
| Serum calcium (mg/dL) | 9.2 ± 0.4 | 9.3 ± 0.4 | <0.001 |
| Serum phosphorus (mg/dL) | 3.5 ± 0.4 | 3.7 ± 0.4 | <0.001 |
| Serum hsCRP (mg/L) | 0.7 ± 1.1 | 1.0 ± 1.3 | <0.001 |
| Serum CA-125 (U/mL) | 9.6 ± 6.4 | 6.0 ± 4.8 | <0.001 |
| Serum CA 19-9 (U/mL) | 13.9 ± 6.9 | 13.3 ± 6.5 | 0.007 |
| Serum CEA (ng /mL) | 1.0 ± 0.6 | 1.4 ± 0.7 | <0.001 |
| Serum AFP (ng/mL) | 2.6 ± 1.4 | 2.9 ± 1.4 | <0.001 |
| BMD (g/cm²) | 0.58 ± 0.3 | 0.54 ± 0.3 | <0.001 |
| T-score | −0.8 ± 0.9 | −1.8 ± 1.1 | <0.001 |

Student's t-test for continuous variables or chi-square test for categorical variables was performed.
The lowest result in each lumbar spine and/or hip BMD was used.
SD, standard deviation;
BMI, body mass index;
ALP, alkaline phosphatase;
hsCRP, high-sensitivity C-reactive protein;
CA-125, carbohydrate antigen 125;
CA 19-9, carbohydrate antigen 19-9;
CBA, carcinoembryonic antigen;
AFP, alpha fetoprotein;
BMD, bone mineral density.

TABLE 2a
Clinical and laboratory features of study subjects according to the quintiles
of serum CA-125 (U/mL) levels in pre- and/or post-menopausal women

| | Pre-menopause (n = 1377) | | | | | |
|---|---|---|---|---|---|---|
| Serum CA-125 (U/mL) | Q1 (n = 279) | Q2 (n = 277) | Q3 (n = 277) | Q4 (n = 270) | Q5 (n = 274) | P^{*} |
| Age (years) | 44.2 ± 5.5 | 42.5 ± 6.4^{†} | 43.0 ± 5.7 | 42.9 ± 5.8 | 43.0 ± 5.7 | NS |
| BMI (kg/m²) | 22.4 ± 2.9 | 21.8 ± 2.9 | 22.3 ± 3.1 | 22.0 ± 2.6 | 22.2 ± 2.8 | NS |
| Physical exercise (number of days/week) | 2.1 ± 1.9 | 2.0 ± 1.9 | 2.1 ± 1.9 | 2.0 ± 1.9 | 1.9 ± 1.8 | NS |
| Current or past smoker (n (%)) | 6 (2.2) | 6 (2.2) | 6 (2.3) | 5 (1.9) | 6 (2.3) | NS |
| Habitual drinker (n (%)) | 53 (19.0) | 51 (18.4) | 54 (19.5) | 53 (19.6) | 53 (19.3) | NS |
| Serum ALP (U/L) | 46.1 ± 12.2 | 45.6 ± 12.2 | 45.0 ± 10.7 | 46.2 ± 12.3 | 45.6 ± 10.5 | NS |
| Serum calcium (mg/dL) | 9.1 ± 0.4 | 9.2 ± 0.4 | 9.2 ± 0.4† | 9.2 ± 0.4 | 9.2 ± 0.4 | 0.002 |
| Serum phosphorus (mg/dL) | 3.6 ± 0.4 | 3.5 ± 0.4 | 3.5 ± 0.4 | 3.5 ± 0.4† | 3.4 ± 0.4† | <0.001 |
| T-score^{b} | −0.9 ± 0.9 | −0.8 ± 0.9 | −0.9 ± 0.8 | −0.7 ± 0.9† | −0.7 ± 0.9† | <0.001 |

| | Post-menopause (n = 2392) | | | | | |
|---|---|---|---|---|---|---|
| | Q1 (n = 481) | Q2 (n = 489) | Q3 (n = 466) | Q4 (n = 487) | Q5 (n = 469) | |
| Age (years) | 61.2 ± 6.7 | 60.7 ± 6.8 | 61.6 + 6.7 | 60.0 ± 6.3^{†§} | 58.7 ± 6.8†‡§¶ | <0.001 |
| BMI (kg/m²) | 23.8 ± 2.9 | 23.8 ± 2.7 | 24.0 ± 2.8 | 24.2 ± 2.9 | 24.0 ± 2.8 | NS |
| Physical exercise (number of days/week) | 1.9 ± 1.4 | 1.7 ± 1.6 | 1.8 ± 1.7 | 1.8 ± 1.8 | 1.8 ± 1.7 | NS |
| Current or past smoker (n (%)) | 6 (1.2) | 8 (1.6) | 7 (1.5) | 8 (1.6) | 7 (1.5) | NS |
| Habitual drinker (n (%)) | 72 (15.0) | 72 (14.7) | 71 (15.2) | 73 (15.0) | 73 (15.6) | NS |
| Serum ALP (U/L) | 63.7 ± 17.6 | 62.6 ± 16.6 | 62.3 ± 16.4 | 60.7 ± 16. 4† | 57.6 ± 16.8†‡§¶ | <0.001 |
| Serum calcium (mg/dL) | 9.2 ± 0.4 | 9.3 ± 0.4† | 9.3 ± 0.4† | 9.4 ± 0.4†‡ | 9.3 ± 0.4† | <0.001 |
| Serum phosphorus (mg/dL) | 3.7 ± 0.4 | 3.7 ± 0.4 | 3.7 ± 0.4 | 3.7 ± 0.4 | 3.6 ± 0.4†‡§¶ | <0.001 |
| T-score | −1.9 ± 1.1 | −1.9 ± 1.0 | −1.9 ± 1.0 | −1.7 ± 1.1†§ | −1.5 ± 1.2†‡§¶ | <0.001 |

TABLE 2b
Clinical and laboratory features of study subjects according to the quintiles
of serum CEA (ng/mL) levels in pre- and/or post-menopausal women

| | Pre-menopause (n = 1377) | | | | | |
|---|---|---|---|---|---|---|
| Serum CEA (ng/mL) | Q1 (n = 278) | Q2 (n = 282) | Q3 (n = 271) | Q4 (n = 313) | Q5 (n = 233) | P^{*} |
| Age (years) | 41.7 ± 6.1 | 42.2 ± 6.2 | 43.7 ± 5.4†‡ | 44.0 ± 5.6†‡ | 44.2 ± 5.4†‡ | <0.001 |
| BMI (kg/m²) | 22.0 ± 2.8 | 22.3 ± 3.0 | 22.3 ± 2.9 | 22.1 ± 2.8 | 22.1 ± 2.8 | NS |
| Physical exercise (number of days/week) | 2.0 ± 1.9 | 2.1 ± 1.9 | 1.9 ± 1.9 | 2.0 ± 1.9 | 1.9 ± 1.7 | NS |
| Current or past smoker (n (%)) | 5 (1.8) | 6 (2.1) | 6 (2.2) | 7 (2.2) | 5 (2.1) | NS |
| Habitual drinker (n (%)) | 53 (19.1) | 54 (19.1) | 52 (19.2) | 57 (18.2) | 48 (20.6) | NS |
| Serum ALP (U/L) | 45.5 ± 13.2 | 46.4 ± 11.5 | 45.3 ± 10.9 | 45.2 ± 10.9 | 46.1 ± 11.3 | NS |
| Serum calcium (mg/dL) | 9.1 ± 0.4 | 9.2 ± 0.4 | 9.2 ± 0.4 | 9.2 ± 0.4† | 9.2 ± 0.4 | 0.021 |
| Serum phosphorus (mg/dL) | 3.5 ± 0.4 | 3.5 ± 0.4 | 3.5 ± 0.4 | 3.5 ± 0.4 | 3.5 ± 0.4 | NS |
| T-score | −1.0 ± 0.8 | −0.9 ± 0.8† | −0.7 ± 0.9† | −0.7 ± 0.9‡ | −0.8 ± 0.9 | <0.001 |

Data are presented as mean ± SD, unless otherwise specified. The quintiles of serum CA-125 and CEA levels were as follows: pre-menopausal CA-125 (Q1, <4.4; Q2, 4.4-6.9; Q3, 6.9-9.6; Q4, 9.6-14.0; Q5, >14.0), post-menopausal CA-125 (Q1, <2.1; Q2, 2.1-4.0; Q3, 4.0-5.7; Q4, 5.7-8.9; Q5, >8.9), and pre-menopausal CEA (Q1, <0.4; Q2, 0.4-0.8; Q3, 0.8-1.1; Q4, 1.1-1.5; Q5, >1.5). Analysis of variance (ANOVA) and multiple comparisons with Tukey method for continuous variables and chi-square test for categorical variables were performed (†, vs Q1; ‡, vs Q2; §, vs Q3; and ¶, vs Q4 presented a statistical significance). The lowest result in each lumbar spine and/or hip BMD was used. CA-125, carbohydrate antigen 125; CEA, carcinoembryonic antigen; BMI, body mass index; ALP, alkaline phospherase; NS, not significant.
*P for trend was calculated for linearity test.

TABLE 3
Age- and BMI-adjusted partial correlation coefficients among serum CA-125, CEA, ALP
and hsCRP levels, and BMD of lumbar spine or femur in pre- and post-menopausal women

| | Serum CA-125 | | Serum CEA | | Serum ALP | | Serum hsCRP | | BMD | |
|---|---|---|---|---|---|---|---|---|---|---|
| | (U/mL) | | (ng/mL) | | (U/L) | | (mg/L) | | (g/cm²) | |
| Variables | r | P | r | P | r | P | r | P | r | P |
| Pre-menopause | | | | | | | | | | |
| Serum CA-125 (U/mL) | 1.000 | — | 0.086 | 0.001 | −0.017 | NS | 0.043 | NS | 0.102 | <0.001 |
| Serum CEA (ng/mL) | — | — | 1.000 | — | 0.016 | NS | −0.013 | NS | 0.134 | <0.001 |
| Serum ALP (U/L) | — | — | — | — | 1.000 | — | 0.130 | <0.001 | −0.197 | <0.001 |
| Serum hsCRP (mg/L) | — | — | — | — | — | — | 1.000 | — | 0.021 | NS |
| BMD (g/cm²) | — | — | — | — | — | — | — | — | 1.000 | — |
| Post-menopause | | | | | | | | | | |
| Serum CA-125 (U/mL) | 1.000 | — | 0.116 | <0.001 | −0.129 | <0.001 | 0.052 | 0.01 | 0.104 | <0.001 |
| Serum ALP (U/L) | — | — | — | — | 1.000 | — | 0.082 | <0.001 | −0.298 | <0.001 |
| Serum hsCRP (mg/L) | — | — | — | — | — | — | 1.000 | — | 0.007 | NS |
| BMD (g/cm²) | — | — | — | — | — | — | — | — | 1.000 | — |

BMI, body mass index; CA-125, carbohydrate antigen 125; CEA, carcinoembryonic antigen; ALP, alkaline phospherase; hsCRP, high sensitivity C-reactvie protein; BMD, bone mineral density; NS, not significant.

TABLE 4
Sensitivity, specificity, positive predictive value (PPV), and negative predictive
value (NPV), and ROC analysis of lower serum CA-125 for osteopenia and/or
osteoporosis in otherwise healthy pre- and post-menopausal women

| Osteopenia/osteoporosis | Sensitivity (%) | Specificity (%) | PPV (%) | NPV (%) | AUC | SE | P |
|---|---|---|---|---|---|---|---|
| Premenopause | | | | | | | |
| Serum CA-125 < 9.6 U/ml | 66.6 | 43.8 | 45.9 | 64.7 | 0.552 | 0.016 | 0.001* |
| Postmenopause | | | | | | | |
| Serum CA-125 < 8.9 U/ml | 83.3 | 29.2 | 79.4 | 34.8 | 0.562 | 0.014 | 0.000* |

ROC, receiver operating characteristic; CA-125, carbohydrate antigen 125; CEA, carcinoembryonic antigen; AUC, area under the curve; and SE, standard error.
*The ROC graphs were not shown.

While the invention has been shown and described with reference to certain exemplary embodiments thereof, it will be understood by those skilled in the art that various changes in form and details may be made therein without departing from the spirit and scope of the invention as defined by the appended claims.

What is claimed is:

1. A kit for diagnosing osteoporosis or osteopenia, or extent of growth plate development comprising a probe selectively binding to carbohydrate antigen 125 (CA-125).

2. The kit of claim 1, wherein the probe is selected from the group consisting of a primer and a nucleic acid probe selectively binding to CA-125.

3. The kit of claim 1, wherein the probe is an antibody selectively binding to CA-125.

4. The kit of claim 1, wherein the kit is to detect expression of CA-125 either at the nucleic acid or protein level.

5. A method for diagnosing osteoporosis or osteopenia, or extent of growth plate development in an individual, said method comprising:
contacting a biological sample from said individual with a probe selectively binding to carbohydrate antigen 125 (CA-125) and determining expression of CA-125,
wherein under-expression of the CA-125, relative to a normal control, is indicative of osteoporosis or osteopenia in the individual; or
if the individual is a child and adolescent, increased level of the CA-125 is indicative of increase of the extent of growth plate development.

6. The method of claim 5, wherein said biological sample is selected from the group consisting of tissues, cells, blood, serum, plasma, sputum, and urine.

7. The method of claim 5, wherein the probe is selected from the group consisting of a primer and a nucleic acid probe selectively binding to CA-125.

8. The method of claim 5, wherein the probe is a plurality of antibodies selectively binding to CA-125.

9. The method of claim 5, wherein the expression of CA-125 is detected either at the nucleic acid or protein level.
